(19) United States
(12) Patent Application Publication
(10) Pub. No.: US 2022/0034899 A1
(43) Pub. Date: Feb. 3, 2022

(54) ANTIBODY QUANTIFICATION IN BIOLOGICAL SAMPLES

(71) Applicant: ICHNOS SCIENCES S.A., La Chaux-de-Fonds (CH)

(72) Inventors: Girish GUDI, La Chaux-de-Fonds (CH); Sunitha GN, La Chaux-de-Fonds (CH); Eric FLUHLER, La Chaux-de-Fonds (CH)

(21) Appl. No.: 17/279,852

(22) PCT Filed: Sep. 25, 2019
(86) PCT No.: PCT/EP2019/075918

§ 371 Date: Mar. 25, 2021

(30) Foreign Application Priority Data
Sep. 25, 2018 (EP) 18196523.7
Sep. 26, 2018 (EP) 18196773.8

(51) Int. Cl. G01N 33/68

(52) U.S. Cl. CPC G01N 33/6848; G01N 33/6854; G01N 2333/71; G01N 2333/82; G01N 2333/70596; G01N 2333/7051

(57) ABSTRACT

The invention relates to a method for quantifying bispecific antibodies, in particular bispecific antibody therapeutics, in biological samples by quantifying a unique signature peptide of said antibody by mass spectrometry. The invention relates also to a kit comprising the unique signature peptide.

### FIELD OF THE INVENTION

The invention relates to a method for quantifying bispecific antibodies, in particular bispecific antibody therapeutics, in biological samples by quantifying a unique signature peptide of said antibody by mass spectrometry. The invention relates also to a kit comprising the unique signature peptide.

### BACKGROUND OF THE INVENTION

With the rapid growth of therapeutic monoclonal antibodies (mAbs) in drug development, quantitative bioanalysis of mAb therapeutics has become essential to support preclinical and clinical studies. Traditionally, Pharmacokinetic analytical methods have employed immunological-based assays for the quantitative analyses of proteins in biological matrices. Immunological-based methods can detect proteins in complex matrices, such as serum, down to the low pg/ml concentration. However, immunological assays require the development of suitable capture and detection reagents, which takes time and resources, and may not be affordable in drug discovery and early development.

Due to their potential advantages (e.g., wide dynamic range, fast method development, reduced need for specific reagents and ability to quantify multiple proteins simultaneously), Mass spectrometry (MS)-based assays have gained interest for mAb quantification in the recent years. For mAbs to be quantified by MS, they first need to be differentiated from the very similar polyclonal background of >1 g/dL of endogenous human immunoglobulins (Igs) in serum. Most mass spectrometry methods rely on the proteolytic digestion of the target mAb and quantification of at least one unique signature peptide which is equivalent to levels of the whole protein. The unique signature peptide for therapeutic mAb quantification in human serum is from the immunoglobulin variable region, which involves the identification and subsequent use of a new signature peptide for each therapeutic mAb.

Liquid chromatography-tandem mass spectrometry (LC-MS/MS) coupled with immunoaffinity sample enrichment is the current method of choice to achieve the most sensitive LC-MS assay for therapeutic (human) mAb quantification in serum, reaching lower limit of quantification (LLOQ) at low ng/level in non-primate mammalian serum (5 ng/ml in rat serum) and higher than 100 ng/ml in human serum. This level of sensitivity is insufficient for pharmacokinetic studies in human or non-human preclinical species, especially for mAbs that are administered at low doses, such as anti-CD3 bispecific antibodies.

Therefore, there is a need for more sensitive LC-MS/MS-based assays for the quantification of therapeutic mAbs in human and non-human primate serum. A highly sensitive LC-MS/MS-based assay that would use the same reagents (signature peptide) for the quantification of various mAbs would be most wanted.

### SUMMARY OF THE INVENTION

The inventors have identified a unique signature peptide for bispecific antibody quantification by mass spectrometry, which allows highly sensitive antibody detection in human serum (LLOQ of 50 pg/ml and detection/quantitation range from 50 pg/ml to 5000 pg/ml). The sensitivity obtained with the signature peptide is suitable for preclinical and clinical pharmacokinetic studies of therapeutic bispecific antibodies administered at low doses such as anti-CD3 bispecific antibodies. The signature peptide, which was not identified using standard prediction rules for selecting signature peptides, is situated in the CH3 domain of bispecific antibodies comprising an engineered human IgG CH3 heterodimer. Therefore, this unique signature peptide can be used advantageously for the highly sensitive and specific quantification of all the bispecific antibodies comprising said engineered CH3 heterodimer, independently of their specificity. The highly sensitive and specific antibody detection combined with the versatility make this new signature peptide a very useful tool for therapeutic bispecific antibodies quantification in preclinical and clinical studies.

Therefore, the invention provides a method for quantifying a bispecific antibody in a biological sample, wherein the antibody comprises an engineered human IgG CH3 heterodimer comprising several substitutions in the CH3 domains including at least two substitutions at positions 80 to 88 of a first CH3 domain, wherein the method comprises quantifying a signature peptide of said antibody by mass spectrometry, wherein the signature peptide is a tryptic peptide corresponding to positions 80 to 88 of said first CH3 domain, and wherein the amino acid positions are indicated according to IGMT® numbering.

In some embodiments of the method according to the invention, the signature peptide consists of a sequence:

(SEQ ID NO: 1)
TX₁PPX₂LX₃SX₄GSFX₅LX₆SX₇

wherein X₁ represents T or D, X₂ represents V, L, P or M, X₃ represents D, Q or E, X₄ represents D or Q, X₅ represents F, A or W, X₆ represents S, W or H, and X₇ represents K or R, with the proviso that when X₁ is T, then at least one of X₂, X₃, X₄, X₅, and X₇ is such that X₂ is L, P or M; X₃ is Q or E; X₄ is Q; X₅ is A or W; and X₇ is R.

In some preferred embodiments, the signature peptide is selected from the group consisting of: TTPPVLDSDGSFALSSK (SEQ ID NO: 3), TDPPLLESDGSFALSSR (SEQ ID NO: 4), TDPPLLESQGSFALSSR (SEQ ID NO: 5), TTPPPLQSDGSFWLWSK (SEQ ID NO: 6) and TTPPMLESDGSFFLHSK (SEQ ID NO: 7), preferably SEQ ID NO: 4 or SEQ ID NO: 5.

In some embodiments, the bispecific antibody comprises a human IgG CH3 domain heterodimer engineered using T-cell receptor-based immunoglobulin domain interface, wherein the first CH3 domain is from human IgG1 and comprises at least the substitutions F85.1A and Y86S and the second CH3 domain is from human IgG1 or IgG3 and comprises at least the substitutions S20K, T22V, K26T, K79Y, K88W and T90N, and wherein said positions are indicated according to IGMT® numbering.

In some preferred embodiments:
the first CH3 domain further comprises one or more of the following substitutions: Q3E, Y5A, L7F, 520T, T22V, K26T, T81D, V84L, D84.2E, K88R and T90R; preferably Q3E, Y5A, L7F, 520T, T22V, K26T, T81D, V84L, D84.2E, K88R and T90R or Q3E, Y5A, L7F, 520T, T22V, K26T, T81D, V84L, D84.2E and K88R; and
the second CH3 domain further comprises one or more of the following substitutions: Q3A, D12E, L14M, N44S, V84M, F85.1S, Y86V, V101I, H115R and Y116F; preferably F85.1S and Y86V or F85.1S, Y86V and Q3A for a CH3 domain from human IgG1, and D12E, L14M, N44S, V84M, F85.1S, Y86V, V101I, H115R and Y116F for a CH3 domain from human IgG3. A bispecific antibody according to these embodiments comprises a signature peptide of SEQ ID NO: 3, 4, 5. Preferably, the bispecific antibody comprises a signature peptide of SEQ ID NO: 4 or 5.

In some other embodiments, the bispecific antibody comprises a human IgG CH3 domain heterodimer engineered using immunoglobulin domain interface exchange between human IgG and IgD CH3 domains, wherein the first CH3 domain comprises the substitutions: Q3V, Y5L, K26S, V84P, D84.2Q, F85.1W and Y86W and the second CH3 domain comprises the substitutions: S20W, K79A, T81A, K88V and T90R. A bispecific antibody according to these embodiments comprises a signature peptide of SEQ ID NO: 6.

In some other embodiments, the bispecific antibody comprises a human IgG CH3 domain heterodimer engineered using immunoglobulin domain interface exchange between human IgG and IgM CH3 domains, wherein the first CH3 domain comprises the substitutions: Q3D, K26T, V84M, D84.2E and Y86H and the second CH3 domain comprises the substitutions: 520T, K79V, T81S and K88I. A bispecific antibody according to these embodiments comprises a signature peptide of SEQ ID NO: 7.

In some embodiments, the bispecific antibody comprises a Fc, a Fab and a scFv from human immunoglobulin, preferably human IgG, more preferably human IgG1 or IgG3.

In some embodiments, the bispecific antibody is a therapeutic antibody, preferably a bispecific anti-CD3 antibody, more preferably anti-CD3 and anti-Her2, anti-CD3 and anti-CD38 or anti-CD3 and anti-EGFR bispecific antibody.

In some embodiments, the method according to the invention comprises the steps of:

a) purifying the bispecific antibody from the biological sample by immunocapture,

b) digesting the bispecific antibody obtained in step a) with trypsin or trypsin/Lys C to generate peptides comprising the signature peptide, and

c) subjecting the peptides obtained in step b) to mass spectrometry to determine the amount of signature peptide in the biological sample by comparison with an internal standard or standard curve.

In some preferred embodiments, the immunocapture is performed with an antigen or antibody specific for the bispecific antibody; preferably an anti-idiotype antibody, an antibody against the signature peptide, or a combination thereof; preferably the immunocapture is performed on a solid support or using immunomagnetic separation; more preferably using a biotinylated antibody and streptavidin coated magnetic beads.

In some embodiments, the mass spectrometry comprises two-dimensional nano-liquid chromatography coupled to electrospray-ionization Orbitrap mass spectrometry.

In other embodiments different mass spectrometry techniques are used.

In some embodiments, the biological sample is a human biological sample, preferably a human body fluid, more preferably human serum.

In some embodiments, the lower limit of quantification (LLOQ) of the bispecific antibody is 50 pg/ml and the detection range of the bispecific antibody is from 50 pg/ml to 5000 pg/ml in human serum.

The invention provides also a kit for performing the method for quantifying a bispecific antibody in a biological sample according to the invention, comprising at least the signature peptide according to the invention; preferably further comprising an antibody specific for the bispecific antibody as defined above.

In particular the kit includes a stable isotope labeled internal standard which has the same sequence or an extended sequence as the signature peptide

### DETAILED DESCRIPTION OF THE INVENTION

The invention provides a highly sensitive and specific method for quantifying a bispecific antibody comprising an engineered human IgG CH3 heterodimer, in a biological sample. The method comprises quantifying a unique signature peptide of said antibody by mass spectrometry, wherein the signature peptide is a tryptic peptide corresponding to positions 80 to 88 of one CH3 domain, wherein the amino acid positions are indicated according to IGMT® numbering.

### Definitions

As used herein, a “bispecific antibody” refers to an antibody comprising an immunoglobulin Fc heterodimer linked to two different antigen-binding domains (or antigen-binding arms), which bind to two different epitopes. The two different antigen-binding domains are part of two different immunoglobulin Heavy chains (Hc) that heterodimerize instead of forming homodimers, through a pair of engineered CH3 domains that form the engineered CH3 heterodimer.

As used herein, “an engineered CH3 heterodimer” refers to a CH3 heterodimer comprising mutations at the interface of the CH3 domains that promote heterodimer assembly and hinder homodimer formation. Various techniques that are well-known in the art can be used for engineering CH3 heterodimers, such as “Knobs-into-Holes” (KiH), “Strand-Exchange Engineered Domain” (SEED) and “Immunoglobulin domain interface exchange”. Immunoglobulin domain interface exchange includes in particular exchanging the homodimer protein-protein interface of an Immunoglobulin (for example, IgG CH3 such as IgG1 CH3 or IgG3 CH3) with a complete heterodimeric interface (T-cell receptor (TCR) α/β or TCR γ/δ constant domain pairs) or half of a homodimeric interface (for example, IgA CH3, IgD CH3, IgGM CH4). Immunoglobulin domain interface exchange is disclosed in WO 2012/131555 and Skegro et al., JBC, 2017, 292, 9745-9759. Bispecific antibodies engineered using TCR-based Immunoglobulin domain interface exchange technology are designated BEAT® antibodies for Bispecific Engagement by Antibodies based on the T-cell receptor. The two CH3 domains of the CH3 heterodimer form at least 60%, preferably at least 70%, 80% or 90% of heterodimers. Heterodimer formation can be measured by standard assays that are known in the art (see for example, Skegro et al., JBC, 2017, 292, 9745-9759).

As used herein, “a signature peptide” refers to a peptide which is unique for the bispecific antibody, which means that it is not found in other proteins of the biological sample.

As used herein, “a biological sample” refers to a complex matrix comprising a mixture of proteins.

The amino acid positions are herein indicated according to IGMT® numbering.

The CH3 domain comprising the signature peptide is herein designated the first CH3 domain.

### Bispecific Antibody and Signature Peptide

The bispecific antibody that is quantified according to the method of the invention comprises a unique signature peptide, which is a tryptic peptide derived from amino acid sequence from positions 80 to 88 of human IgG CH3 domain:

(SEQ ID NO: 2)
TTPPZ₁LDSDGSFFLYSZ₂,

wherein Z₁ is V or M and Z₂ is K or R, and
wherein said signature peptide comprises at least two amino acid substitutions compared to SEQ ID NO: 2.

The signature peptide can comprise 2, 3, 4, 5, 6, 7 or more amino acid substitutions in SEQ ID NO: 2, preferably, 6, 7 or more amino acid substitutions.

Since the signature peptide is a tryptic peptide, the antibody sequence comprises a lysine (K) or arginine (R) in position-1 relative to the signature peptide sequence.

The signature peptide, which is a unique peptide, is found only in the first CH3 domain (i.e. not in the second CH3 domain).

In some preferred embodiments, the signature peptide consists of a sequence:

(SEQ ID NO: 1)
TX₁PPX₂LX₃SX₄GSFX₅LX₆SX₇,

wherein: X₁ represents T or D,
   X₂ represents V, L, P or M,
   X₃ represents D, Q or E,
   X₄ represents D or Q,
   X₅ represents F, A or W,
   X₆ represents S, W or H, and
   X₇ represents K or R,
      with the proviso that when X₁ is T, then at least one of X₂, X₃, X₄, X₅, and X₇ is such that X₂ is L, P or M; X₃ is Q or E; X₄ is Q; X₅ is A or W; and X₇ is R. Preferably, at least two, three, four, or all of X₂, X₃, X₄, X₅, and X₇ are such that X₂ is L, P or M; X₃ is Q or E; X₄ is Q; X₅ is A or W; and X₇ is R; more preferably, four or all of X₂, X₃, X₄, X₅, and X₇ are such that X₂ is L, P or M; X₃ is Q or E; X₄ is Q; X₅ is A or W; and X₇ is R.

In some more preferred embodiments, the signature peptide is selected from the group consisting of: TTPPVLDSDGSFALSSK (SEQ ID NO: 3), TDPPLLESDGSFALSSR (SEQ ID NO: 4), TDPPLLESQGSFALSSR (SEQ ID NO: 5), TTPPPLQSDGSFWLWSK (SEQ ID NO: 6) and TTPPMLESDGSFFLHSK (SEQ ID NO: 7); preferably SEQ ID NO: 4 or SEQ ID NO: 5.

The bispecific antibody that is quantified according to the method of the invention comprises an immunoglobulin Fc heterodimer comprising an engineered human IgG CH3 heterodimer, wherein the CH3 heterodimer comprises several substitutions in the CH3 domains, including at least two substitutions at positions 80 to 88 of the first CH3 domain.

In the various embodiments of the method, the engineered CH3 heterodimer is from human IgG1, IgG2, IgG3, IgG4 or combination thereof. The first CH3 domain is preferably from human IgG1, IgG2 or IgG4, more preferably from human IgG1. The second CH3 domain is from any human IgG. In some preferred embodiments, the first CH3 domain is from human IgG1 and the second CH3 domain is from human IgG1 or IgG3.

In the various embodiments of the method, the engineered CH3 heterodimer comprises 2, 3, 4, 5, 6, 7 or more substitutions at positions 80 to 88 of the first CH3 domain, preferably, 6, 7 or more substitutions.

In some embodiments, the first CH3 domain of the engineered CH3 heterodimer is from human IgG1 and the second CH3 domain is from human IgG1 or IgG3, and the first CH3 domain comprises 6, 7, or more substitutions at positions 80 to 88.

In some preferred embodiments, the bispecific antibody comprises a human IgG CH3 domain heterodimer engineered using the TCR-based Immunoglobulin domain interface exchange (BEAT®) technology, wherein the first CH3 domain is from human IgG1 and comprises at least the substitutions F85.1A and Y86S and the second CH3 domain is from human IgG1 or IgG3 and comprises at least the substitutions S20K, T22V, K26T, K79Y, K88W and T90N.

In some more preferred embodiments:
the first CH3 domain further comprises one or more of the following substitutions: Q3E, Y5A, L7F, S20T, T22V, K26T, T81D, V84L, D84.2E, K88R and T90R; preferably Q3E, Y5A, L7F, S20T, T22V, K26T, T81D, V84L, D84.2E, K88R and T90R or Q3E, Y5A, L7F, S20T, T22V, K26T, T81D, V84L, D84.2E and K88R; and
the second CH3 domain further comprises one or more of the following substitutions: Q3A, D12E, L14M, N44S, V84M, F85.1S, Y86V, V101I, H115R and Y116F; preferably F85.1S and Y86V or F85.1S, Y86V and Q3A for a CH3 domain derived from human IgG1; and D12E, L14M, N44S, V84M, F85.1S, Y86V, V101I, H115R and Y116F for a CH3 domain derived from human IgG3.

A bispecific antibody according to the first preferred embodiments comprises a signature peptide of SEQ ID NO: 3, 4, 5. Preferably, the bispecific antibody comprises the signature peptide of SEQ ID NO: 4 or 5.

In some other preferred embodiments, the bispecific antibody comprises a human IgG1 CH3 domain heterodimer engineered using immunoglobulin domain interface exchange between human IgG and IgD CH3 domains, wherein the first CH3 domain comprises the substitutions: Q3V, Y5L, K26S, V84P, D84.2Q, F85.1W and Y86W and the second CH3 domain comprises the substitutions: S20W, K79A, T81A, K88V and T90R. A bispecific antibody according to these embodiments comprises a signature peptide of SEQ ID NO: 6.

In some yet other preferred embodiments, the bispecific antibody comprises a human IgG1 CH3 domain heterodimer engineered using immunoglobulin interface exchange between human IgG and IgM CH3 domains, wherein the first CH3 domain comprises the substitutions: Q3D, K26T, V84M, D84.2E and Y86H and the second CH3 domain comprises the substitutions: S20T, K79V, T81S and K88I. A bispecific antibody according to these embodiments comprises a signature peptide of SEQ ID NO: 7.

The bispecific antibody that is quantified according to the method of the invention comprises an immunoglobulin Fc heterodimer, linked to two different antigen-binding domains (or antigen-binding arms) which bind to two different epitopes.

The Fc heterodimer comprises at least the engineered human IgG CH3 heterodimer. It usually further comprises at least a pair of CH2 homodimers, preferably from IgG, more preferably from human IgG, still more preferably from human IgG1.

The Fc heterodimer is advantageously linked to the antigen-binding arms via two immunoglobulin hinges, preferably IgG hinges, more preferably human IgG hinges, still more preferably human IgG1 hinges.

The antigen-binding arms can be immunoglobulin Fab or scFv fragments, preferably human or humanized Fab or scFv fragments. The bispecific antibody can comprise two Fab fragments, two scFv fragments or one Fab fragment and one scFv fragment.

In some embodiments, the bispecific antibody comprises a Fc heterodimer, a Fab and a scFv from human immunoglobulin. The Fc heterodimer is preferably from human IgG1. The Fc heterodimer is preferably linked to each of the Fab and scFv through a IgG1 hinge, preferably a human IgG1 hinge.

In some embodiments, the bispecific antibody is a therapeutic antibody. The bispecific antibody is directed to two therapeutic targets. Non-limiting examples of therapeutic targets of the bispecific antibody include: CD3, CD38, Her-2, EGFR, CD20, TNFα, VEGF, CEA, IL-12, IL-23, PD-L1, PD-1, complement C5, and others. Numerous therapeutic targets for monoclonal antibodies are well-known in the art and numerous monoclonal antibodies directed to various targets are available for the treatment of various diseases such as cancer, auto-immune, inflammatory diseases, infectious diseases and other diseases. The bispecific antibody can be directed to any of these therapeutic targets or can be derived from any of these therapeutic monoclonal antibodies.

In some preferred embodiments, the therapeutic bispecific antibody is a bispecific anti-CD3 antibody, preferably anti-CD3 and anti-Her2, anti-CD3 and anti-CD38 or anti-CD3 and anti-EGFR bispecific antibody.

### Biological Sample Preparation

The biological sample is preferably from a human or animal source that has been treated with the bispecific antibody, more preferably a human or simian subject, still more preferably a human subject. The sample is a biological tissue or fluid, preferably a biological fluid, such as with no-limitations: whole-blood, serum, plasma, urine, tissue biopsies or mucosal secretion (saliva, lachrymal fluid, broncho-alveolar lavage fluid and others), more preferably plasma or serum.

The sample can be treated using conventional techniques to extract antibodies and/or remove interfering components. For example solid and/or tissue samples can be homogenized and centrifuged, filtered, and/or subjected to chromatographic techniques to remove cells or tissue fragments. In other cases, reagents known to precipitate or bind the interfering components can be added. For example, whole-blood can be treated using conventional clotting techniques to remove red and white blood cells and platelets.

Bispecific antibodies can be isolated from the samples or enriched (i.e. concentrated) in a sample using standard methods used for monoclonal antibodies that are known in the art. Such methods include removing one or more non-bispecific antibody contaminants from a sample. The samples can be enriched or purified using centrifugation, filtration, ultrafiltration, dialysis, ion exchange chromatography, size exclusion chromatography, protein A/G affinity chromatography, affinity purification, precipitation, gel electrophoresis, capillary electrophoresis and chemical fractionation. In some embodiments, the bispecific antibody, or the heavy and/or light chains thereof are substantially isolated, which means that the bispecific antibody is at least partially or substantially separated from the sample from which it was provided. Substantial separation can include samples containing at least about 10%, at least about 20%, at least about 30%, at least about 40%, at least about 50%, at least about 60%, at least about 70%, at least about 80%, at least about 90%, at least about 95%, at least about 97%, or at least about 99% by weight of the bispecific antibody, or the heavy and/or light chains thereof. Methods for isolating monoclonal antibodies, such as those described above, are routine in the art.

In some preferred embodiment, the bispecific antibody is purified from the biological sample by immunocapture. The immunocapture is performed with an antigen or antibody specific for the bispecific antibody; preferably an anti-idiotype antibody, an antibody against the signature peptide, or a combination thereof, wherein the anti-idiotype and anti-peptide antibodies are used successively. When the bispecific antibody is an anti-CD3 antibody, the immunocapture is performed using an anti-CD3 idiotype antibody, for example an anti-OKT3 antibody. The immunocapture is preferably performed on a solid support or by using immunomagnetic separation; more preferably using biotinylated antibody and streptavidin coated magnetic beads. Immunocapture is performed using standard methods used for monoclonal antibodies that are known in the art.

Following purification, preferably by immunocapture, the bispecific antibody is digested with trypsin or Trypsin/LysC to generate (tryptic) peptides comprising the signature peptide. Trypsin or Trypsin/LysC digestion is performed using standard methods that are well-known in the art. The digestion conditions (incubation time, temperature, trypsin to bispecific antibody ratio) are optimized to ensure sufficient and consistent digestion. Immobilized trypsin or trypsin/lysC is advantageously used.

A pretreatment is advantageously performed to improve digestion efficiency and completeness by unfolding the bispecific antibody, reducing the disulfide bonds between the heavy and light chains and preventing their reformation. This can be achieved by treating the bispecific antibody with a reducing agent such as DTT and DTE (2,3 dihydroxybutane-1,4-dithiol), thioglycolate, cysteine sulfites, bisulfites, sulfides, bisulfides, TCEP (tris(2-carboxyethyl)phosphine) and 2-mercaptoethanol, and an alkylation agent such as iodoacetamide. Additional treatment with a denaturing agent such as urea can be performed before treatment with the reducing agent. Alternatively, tryptic digestion process can be accelerated by elevated digestion temperature, addition of organic solvent, microwave-assisted digestion or pellet digestion methodology. For example, the bispecific antibody can be treated with TCEP and iodoacetamide.

### Mass Spectrometry

After sample preparation, the tryptic peptides of the bispecific antibody are subjected to tandem mass spectrometry (MS) technique (LC-MS/MS) which combines the physical separation capabilities of liquid chromatography with the mass analysis capabilities of mass spectrometry. Mass spectrometry analysis relies on the separation of charged species on the basis of their mass. Any LC-MS instrument can be used. LC is performed using high performance liquid chromatography column. LC is advantageously performed using two-dimensional-high-performance liquid chromatography (2-D-HPLC), such as two-dimensional nano-liquid chromatography. For example, a 2D Trap-Nano LC configuration can be used. The Trap column is advantageously a reverse-phase C18 liquid chromatography HPLC column.

Mass spectrometry detection can be performed using electrospray ionization coupled to a quadrupole mass spectrometer (ESI Triple Quad MS). A quadrupole mass analyzer (Q) consists of four cylinder rods, set parallel to each other. The Q may also consist of other polygonal rods such as hexagonal and octagonal as well as slightly off-parallel set ups. In a quadrupole mass spectrometer, the quadrupole is the component of the instrument responsible for filtering sample ions based on their mass-to-charge ratio (m/z). Any ESI Triple Quad mass spectrometer can be used. Improved sensitivity and specificity can be achieved by using High-resolution accurate-mass spectrometry (HRMS). Examples of HRMS instruments are Orbitrap and Time-Of-Flight (TOF) mass spectrometers.

In some embodiments, the mass spectrometry comprises two-dimensional nano-liquid chromatography coupled to electrospray-ionization Orbitrap mass spectrometry.

The amount of signature peptide in the biological sample is determined by comparison with an internal standard (IS). The internal standard can be a stable-isotope-labeled (SIL) analog of the bispecific antibody, a stable-isotope-labeled (SIL) signature peptide, or a similar bispecific antibody such as one comprising the same signature peptide. The method can achieve a Lower Level of Quantification (LLOQ) of 50 pg/ml and a detection range from 50 pg/ml to 5000 pg/ml of bispecific antibody in human serum.

### Kit

The invention relates also to a kit for quantifying a bispecific antibody in a biological sample using the method according to the invention, comprising at least a signature peptide according to the invention. Preferably, the kit further comprises an antibody specific for the bispecific antibody and/or an internal standard according to the invention.

### Use of the Method

The invention relates also to the use of the method of the invention for performing pharmacokinetic studies of therapeutic bispecific antibodies, in particular preclinical or clinical studies of therapeutic bispecific antibodies, and for monitoring the treatment of a disease with a therapeutic bispecific antibody in a subject.

The present invention relates to T-cell redirecting antibodies, such as bispecific antibody, for use in the treatment of HER2-positive solid cancer.

Also provided by the present disclosure is a method for treating HER2-positive solid cancer by administering to a patient a therapeutically effective amount of the disclosed antibody.

According to one aspect of the present invention, the T-cell redirecting antibody is generated by BEAT® technology (WO2012131555). In a more specific aspect of the present invention the T-cell redirecting antibody is a HER2×CD3 bispecific antibody, known as GBR1302, that redirects cytotoxic T-cells to kill HER2 overexpressing cancer cells. More specifically the antibody of the present invention comprises the amino acid sequences of SEQ ID NOs: 11 to 13. In another specific aspect the T-cell redirecting antibody is a CD38×CD3 bispecific antibody, known as GBR1342 (SEQ ID NOs: 14 to 16).

In accordance with a preferred aspect of the present invention the method is performed using the following parameters:

| S. No | Parameters | Method | |
|---|---|---|---|
| 1 | Elution of antibody from streptavidin beads | 2 × 70 uL of 30 mM HCL with 5% | |
| | | ACN | |
| 2 | Mixing time for acid elution | 10 | min |
| | | Non-winged stable Isotope | |
| | | labelled signature peptide of GBR | |
| 3 | Internal standard (IS) | 1302 | |
| 4 | Concentration of IS | 75 | pg/mL |
| 5 | Calibration range | 50-5000 pg/ml | |
| | | Std 2-7: 100, 200, 400, 800, | |
| 6 | Change in concentration of Standards (Std 2-7) | 1600, 3200 pg/mL | |
| 7 | Arrest of trypsin digestion | Use of Formic acid | |
| | Centrifugation of samples before loading into | | |
| 8 | Nano LC | Done | |
| 9 | Injection volume | 80 | uL |

| | | |
|---|---|---|
| 10 | Left pump flow rate | 0.3 mL/min at 0, 6, 10.1 and 12 |
| | | min retention time |
| 11 | Nano pump mobile phase B composition | 18% at 0, 3, 11 and 12 min of |
| | | retension time |
| 12 | Retention time GBR 1302 and GBR 1302 IS | 3.5 min ± 1 min |
| 13 | MS acquisition time | 3 min to 4 min |
| | | Primary carry over for analyte |
| | | < 500% relative to LLOQ; |
| 14 | Carryover Evaluation | Secondary carry over < 25% |
| | | relative to LLOQ; |

In one embodiment of the present invention, the disclosed T cell redirecting antibody is used for the treatment of an HER2-positive solid tumor.

In a specific embodiment of the present invention, the disclosed antibody is administered intravenously at a dose between 1 ng/kg and 750 ng/kg on Day 1 and on Day 15 in 28-day treatment cycles.

In a more specific embodiment, the treatment dose is selected from the group comprising about 1 ng/kg, about 3 ng/kg, about 10 ng/kg, about 30 ng/kg, about 60 ng/kg, about 100 ng/kg, about 200 ng/kg, about 300 ng/kg, about 500 ng/kg and about 750 ng/kg.

In order to use the disclosed antibody as therapeutic it is necessary to study the time course of drug absorption, distribution, metabolism, and excretion to enhance the efficacy of the drug and decrease its toxicity. To carry on these studies, collectively known as pharmacokinetics studies, certain parameters need to be investigated including: maximum observed serum concentration (Cₘₐₓ), area under the serum concentration time curve from time 0 to time of the last measurable concentration (AUCₗₐₛₜ), time of maximum observed serum concentration (Tₘₐₓ), time of last observed serum concentration (Tₗₐₛₜ), serum elimination half-life (t_{1/2}), last measurable plasma concentration (Cₗₐₛₜ).

According to one aspect of the present invention, the disclosed antibody is administered at a dose of:
(a) about 30 ng/kg, Cₘₐₓ is equal to or greater than about 0.25 ng/mL and equal to or less than about 0.45 ng/mL, AUCₗₐₛₜ is equal to or greater than about 10 hr*ng/mL and equal to or less than about 25 hr*ng/mL, Tₘₐₓ is equal to or greater than about 1 hr and equal to or less than about 4 hr, Tₗₐₛₜ is equal to or greater than about 40 hr and equal to or less than about 160 hr, t_{1/2} is about 70 hr and Cₗₐₛₜ is s equal to or greater than about 0.05 ng/mL and equal to or less than about 0.15 ng/mL;
(b) about 60 ng/kg, Cₘₐₓ is equal to or greater than about 0.3 ng/mL and equal to or less than about 0.9 ng/mL, AUCₗₐₛₜ is equal to or greater than about 1.3 hr*ng/mL and equal to or less than about 90 hr*ng/mL, Tₘₐₓ is equal to or greater than about 1 hr and equal to or less than about 4 hr, Tₗₐₛₜ is equal to or greater than about 4 hr and equal to or less than about 350 hr, t_{1/2} is equal to or greater than about 90 hr and equal to or less than about 130 hr and Cₗₐₛₜ is s equal to or greater than about 0.05 ng/mL and equal to or less than about 0.65 ng/mL;
(c) about 100 ng/kg, Cₘₐₓ is equal to or greater than about 0.5 ng/mL and equal to or less than about 3 ng/mL, AUCₗₐₛₜ is equal to or greater than about 25 hr*ng/mL and equal to or less than about 210 hr*ng/mL, Tₘₐₓ is equal to or greater than about 1 hr and equal to or less than about 5 hr, Tₗₐₛₜ is equal to or greater than about 110 hr and equal to or less than about 360 hr, t_{1/2} is equal to or greater than about 80 hr and equal to or less than about 130 hr and Cₗₐₛₜ is s equal to or greater than about 0.05 ng/mL and equal to or less than about 0.2 ng/mL;
(d) about 200 ng/kg, Cₘₐₓ is equal to or greater than about 0.9 ng/mL and equal to or less than about 2.5 ng/mL, AUCₗₐₛₜ is equal to or greater than about 74 hr*ng/mL and equal to or less than about 230 hr*ng/mL, Tₘₐₓ is equal to or greater than about 1 hr and equal to or less than about 7 hr, Tₗₐₛₜ is equal to or greater than about 140 hr and equal to or less than about 340 hr, t_{1/2} is equal to or greater than about 80 hr and equal to or less than about 130 hr and Cₗₐₛₜ is s equal to or greater than about 0.05 ng/mL and equal to or less than about 0.3 ng/mL;
(e) about 300 ng/kg, Cₘₐₓ is equal to or greater than about 2 ng/mL and equal to or less than about 4.5 ng/mL, AUCₗₐₛₜ is equal to or greater than about 9 hr*ng/mL and equal to or less than about 330 hr*ng/mL, Tₘₐₓ is equal to or greater than about 1 hr and equal to or less than about 6 hr, Tₗₐₛₜ is equal to or greater than about 4 hr and equal to or less than about 540 hr, t_{1/2} is equal to or greater than about 80 hr and equal to or less than about 120 hr and Cₗₐₛₜ is s equal to or greater than about 0.08 ng/mL and equal to or less than about 2.4 ng/mL;
(f) about 500 ng/kg, Cₘₐₓ is equal to or greater than about 2.5 ng/mL and equal to or less than about 8 ng/mL, AUCₗₐₛₜ is equal to or greater than about 160 hr*ng/mL and equal to or less than about 760 hr*ng/mL, Tₘₐₓ is equal to or greater than about 1 hr and equal to or less than about 11 hr, Tₗₐₛₜ is equal to or greater than about 48 hr and equal to or less than about 500 hr, t_{1/2} is equal to or greater than about 100 hr and equal to or less than about 150 hr and Cₗₐₛₜ is s equal to or greater than about 0.1 ng/mL and equal to or less than about 2 ng/mL;
(g) about 7,5 ng/kg, Cₘₐₓ is equal to or greater than about 7.5 ng/mL and equal to or less than about 17 ng/mL, AUCₗₐₛₜ is equal to or greater than about 240 hr*ng/mL and equal to or less than about 1100 hr*ng/mL, Tₘₐₓ is equal to or greater than about 1 hr and equal to or less than about 6 hr, Tₗₐₛₜ is equal to or greater than about 40 hr and equal to or less than about 340 hr, t_{1/2} is equal to or greater than about 100 hr and equal to or less than about 160 hr and Cₗₐₛₜ is s equal to or greater than about 0.35 ng/mL and equal to or less than about 3.5 ng/mL.

In accordance with one aspect of the present invention the T cell redirecting antibody is suitable for treating a cancer characterized by the overexpression of HER2 and in particular selected from the group breast, ovarian, bladder, salivary gland, endometrial, pancreatic and non-small-cell lung cancer (NSCLC). In a favorite aspect of the present invention the HER2-positive cancer is breast cancer.

### FIGURE LEGENDS

FIG. 1 represents LC-HRMS/MS profile of GBR 1302 tryptic digest.

A. GBR 1302 tryptic digest showing peptide TDPPLLESDGSFALSSR (SEQ ID NO: 4) with m/z 896.44 (M+2H)²⁺ B. GBR 1302 tryptic digest showing peptide EPEVATFPPSR (SEQ ID NO: 10) with m/z 615.31 (M+2H)²⁺. C. Human serum blank.

FIG. 2 represents GBR 1302 quantification in human serum by LC-HRMS/MS.

FIG. 3 represents GBR 1342 quantification in monkey serum by LC-HRMS/MS.

FIG. 4: Geometric mean serum profile for GBR1302.

### EXAMPLES

### Materials and Methods

### 1. Materials

Bispecific Antibodies
GBR 1302: anti-CD3/anti-Her2 human IgG1 BEAT® bispecific antibody
GBR 1342: anti-CD3/anti-CD38 human IgG1 BEAT® bispecific antibody
GBR 1372: anti-CD3/anti-EGFR human IgG1 BEAT® bispecific antibody

Internal Standard
GBR 1302 IS: Stable Isotope labelled (SIL) signature peptide SEQ ID NO: 4 (m/z 901.44 (M+2H)²⁺). Working solution 100 pg/mL in 30:70 ACN:Water
GBR 1342 IS: Stable Isotope labelled (SIL) signature peptide SEQ ID NO: 5 (m/z 907.96 (M+2H)²1; Working solution 100 pg/mL in 30:70 ACN:Water

Reagents
Biological matrix: human or monkey serum
Streptavidin Beads (DynaBeads Streptavidin T1, p/n 650-02, Invitrogen)
Biotinylated OKT3 antibody (Abpro and inhouse)
Biotinylated 9G7 antibody (Abpro and inhouse)
Biotinylated SP34 antibody (Bio-rad and inhouse)
TCEP Reducing Solution (75 mM TCEP in water, freshly prepared)
Iodoacetamide Alkylation (IAA) Solution (150 mM IAA in water, freshly prepared)
Trypsin Solution (Trypsin Gold (PROMEGA) 50 μg/mL in water; freshly prepared)
Acetonitrile (ACN) solution: 30:70 ACN:Water
PBS/BSA: 0.1% BSA in 10 mM PBS
TrisHC1, 1M, pH 8.3
HCl 30 mM

Calibration Standards and Quality Control (QC) Samples
QC samples: 50, 150, 300, 750, 4000 pg/mL of bispecific antibody in biological matrix
Standards: STD1 to STD8: 50, 75, 100, 250, 500, 1000, 3000 and 5000 pg/mL of bispecific antibody in biological matrix

### 2. Methods

### 2.1 Sample Preparation

Samples, quality controls, standards, zero samples and blanks in PBS-BSA are distributed in wells of Plate A, biotinylated antibody (2 μg) is then added, and Plate A is incubated overnight at 4° C. with shaking. Streptavidin T1 beads are then added in the wells and Plate A is incubated for 1 h at room temperature to bind up biotinylated-antibody captured analyte. Beads are then transferred to a collection plate (Plate B) placed on a magnetic stand and washed twice with CHAPs buffer and twice with PBS buffer. Antibody elution is performed by adding 30 mM HCl into Plate B, mixing for 3 min and transferring the eluate into Plate C containing 1 M Tris pH 8.3; and repeating the elution step.

Internal standard is added to samples, quality controls, standards and zero samples of Plate C. 30:70 ACN:Water is added to control blanks of Plate C. 75 mM TCEP is added to each well, after mixing for 1 min, the plate is incubated at 56° C. for 45 min. The plate is cooled down at RT for 10 min. 150 mM IAA is added to each well, and after mixing for 1 min, the plate is incubated at RT for 35 min in the dark. Trypsin (50 μg/mL) is added to each well, and after mixing for 1 min, the plate is incubated at 37° C., overnight with shaking.

### 2.3 Chromatography

| Autosampler | Dionex ultimate 3000 AS |
|---|---|
| Injection vol | 100 μL |
| Loading and micro pump | Dionex. |
| | Flow rate: |
| | Loading pump: 50 to 400 ul/min (flow gradient) |
| | Micro pump: 300 to 400 μL/min (flow gradient) |
| Nano LC pump | Dionex ultimate 3000 (NCS-3500RS) nano |
| | pump |
| | Flow rate: 600 nL/min |
| Trap Column | μ-Precolumn Cartridge P/N 160454 |
| | fitted with an Thermo Acclaim PepMap100, |
| | C18 300 μm × 5 mm, 5 μm |
| Analytical Column | Thermo Acclaim PepMap C18 |
| | 75 μm × 15 cm, 5 μm |
| Column Temperature | 60° C. |
| Mobile Phase: Nano | A: 98% H₂O, 2% ACN with |
| | 0.1% formic acid |
| | B: 90% ACN, 10% water with |
| | 0.1% formic acid |
| Mobile Phase: | (1) C: 60% IPA, 30% ACN, 10% water with |
| (1) Loading | 1% formic acid |
| (2) Micro pump | (2) A: 60% IPA, 30% ACN, 10% water with |
| | 1% formic acid B: 1% formic acid in water |

Retention time: GBR 1302 4.40±1.0 minutes
   GBR 1302-IS 4.40±1.0 minutes
   GBR 1342 4.10±1.0 minutes
   GBR 1342-IS 4.10±1.0 minutes

The method is performed using 2D Trap-Nano LC Configuration in which Thermo QE and Dionex ultimate 3000 RSLC nano LC are coupled with Thermo Easy-Spray source. The samples are first loaded by loading pump onto a trap column followed by switching to a nanoLC analytical column operated at a flow rate of 600 nL/min by a nano pump. The analytical column coiled into a loop is intimately coupled with a linear restrictor emitter. The trap column and analytical column are both washed with high organic solvent to elute highly retained endogenous components by nano pump and micropump.

### 2.4 Mass Spectrometry

| Mass Spectrometry | Thermo Q-Exactive Series mass spec | |
|---|---|---|
| Ion mode | Positive ion mode | |
| Experiment Ionization | PRM (Target MS2) | |
| | Thermo Easy Spray | |
| Compounds for inclusion list: | GBR1302: m/z 896.441 z = +2 | |
| | GBR1302 IS: m/z 901.445 z = +2 | |
| | GBR1342: m/z 902.96 z = +2 | |
| | GBR1302 IS: m/z 907.96 z = +2 | |
| Collision energy | 21 | |
| Spray Voltage | 2300 | V |
| Orbitrap Resolution | 17,500 | |
| AGC Target | 5 e5 | |
| Max Injection Time | 50 | ms |

| | |
|---|---|
| S-lens RF amplitude | 90 |
| Capillary Tem. | 275° C. |
| Fragment (charge state z = +2) | GRB1302: m/z 788.404 |
| | GRB1302 IS: m/z 793.408 |
| | GRB1342: m/z 794.919 |
| | GRB1342 IS: m/z 799.923 |

MS Acquisition time: GRB1302 and GRB1302 IS: 4.1 min to 5.1 min
   GRB1342 and GRB1342 IS: 4 min to 5 min

### Example 1: Identification of a Unique Signature Peptide for Bispecific Antibody Quantification in Human or Non-Human Primate Serum

In a first approach, potential signature peptides for quantifying bispecific antibodies in human serum were selected using standard rules for selection: 6-15 aa; no chemical chemical reactive residues (Tryptophan (W), Methionine (M), Cysteine (C)); no inclusion of 2R, 2K and RK; no potential PTM (Tyrosine (Y), Threonine (T), Serine(S), Lysine (K)); preferably containing Proline (P); R in P proximity (potential missed tryptic cleavage). Based on these rules, 15 signature peptides (SPs) were selected in human serum spiked with GBR 1302. Two peptides LYSGVPSR (SEQ ID NO: 8) and FTISADTSK (SEQ ID NO: 9) were selected for optimization based on their mass response intensities. It was observed that the selected signature peptides were present in the blank human serum as well as in GBR 1302 spiked human serum sample suggesting that they were not specific for GBR 1302.

In a second approach, the software Expasy peptide cutter (http://web.expasy.org/peptide_cutter/) was used to predict the peptides generated by Trypsin digestion of GBR 1302, GBR 1342 and GBR 1372 bispecific antibodies. The resulting tryptic peptide sequences were then compared to the human plasma proteome (NCBI BLAST) to exclude peptides which were not unique to the bispecific antibodies (present in the plasma proteome).

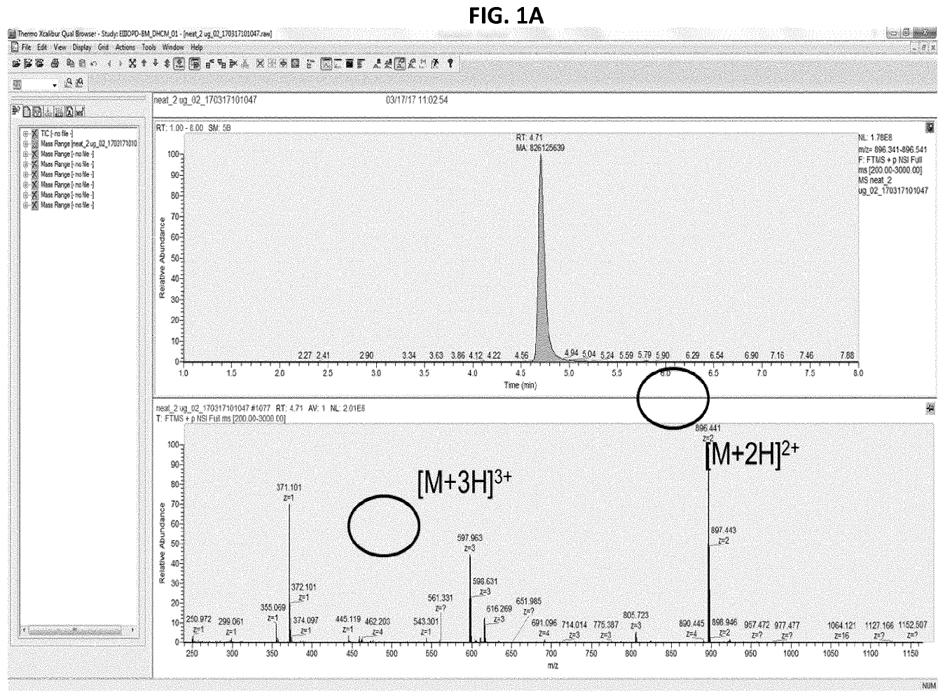

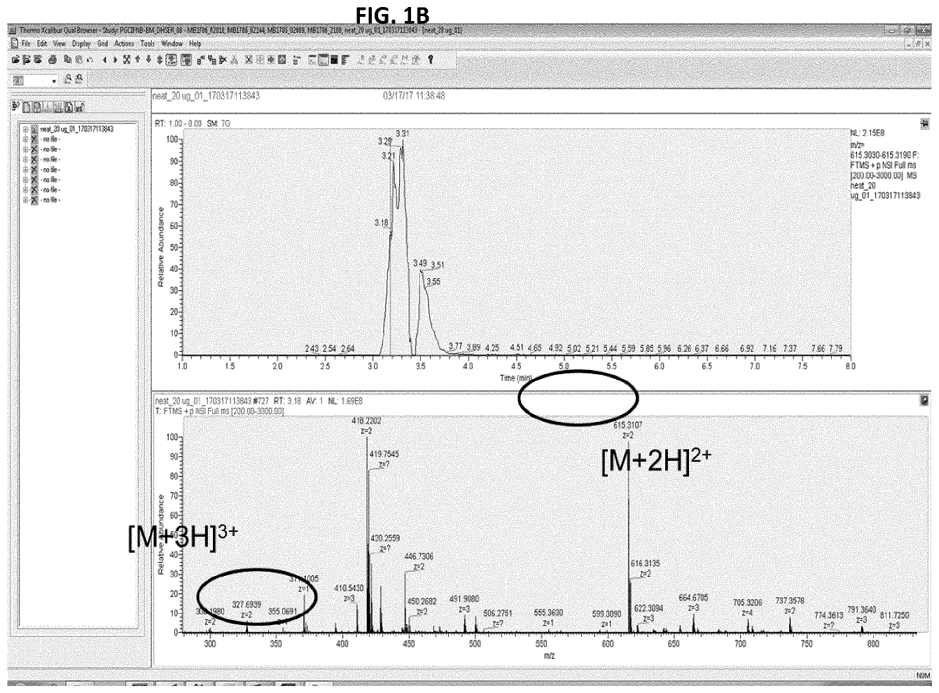

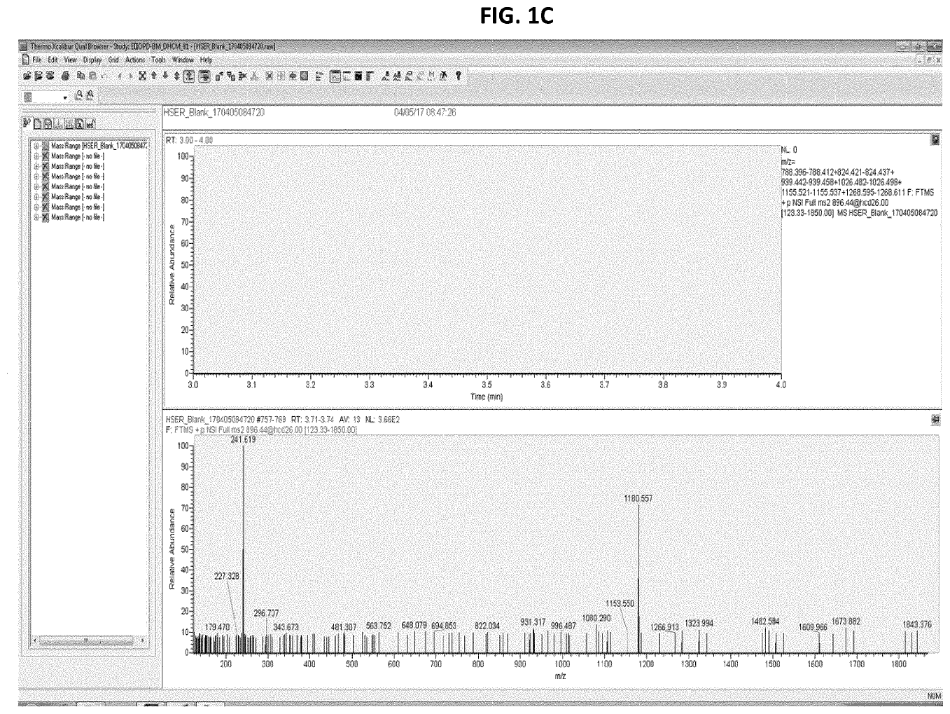

Two unique signature peptides were found in LC-HRMS/MS profile of GBR 1302 tryptic digest: TDPPLLESDGSFALSSR (SEQ ID NO: 4) with m/z 896.44 (M+2H)²⁺ and EPEVATFPPSR (SEQ ID NO: 10) with m/z 615.31 (M+2H)²⁺ (FIGS. 1A and 1B). It was observed that they were unique for GBR 1302 and not present in blank human serum and Trastuzumab (FIG. 1C).

It was also observed that both peptides were situated in the engineered CH3 heterodimer which is present in all the bispecific antibodies generated by immunoglobulin domain interface exchange technology. The SP of SEQ ID NO: 4 is situated from positions 80 to 88 of IgG CH3 domain according to IGMT numbering. The SP of SEQ ID NO: 10 is situated from positions 1 to 11 of IgG CH3 domain according to IGMT numbering.

Based on mass response intensity, signature peptide TDPPLLESDGSFALSSR (SEQ ID NO: 4) was selected for bispecific antibody quantification.

GBR 1372 comprises the same Fc heterodimer as GBR 1302. The same unique signature peptide (SEQ ID NO: 4) was found in LC-HRMS/MS profile of GBR 1372 tryptic digest.

GBR 1342 comprises a Fc heterodimer which differs from that of GBR 1302 and GBR 1372 by a D84.4Q substitution. The corresponding signature peptide TDPPLLESQGSFALSSR (SEQ ID NO: 5) with m/z 902.96 (M+2H)²⁺ was found in LC-HRMS/MS profile of GBR 1342 tryptic digest.

These results show that the signature peptide from positions 80 to 88 of one CH3 domain of an engineered human IgG CH3 heterodimer is a unique signature peptide which can be used for the quantification of all the bispecific antibodies having an engineered human IgG CH3 heterodimer in human serum.

### Example 2: High Sensitivity LC-HRMS/MS Assay for Bispecific Antibody Quantification in Human or Monkey Serum

A LC-HRMS/MS assay based on the detection of the unique signature peptide identified in example 1 was developed for bispecific antibody quantification in human or non-human primate serum. GBR 1302 was quantified in human serum based on MS analysis of the signature peptide SEQ ID NO: 4. GBR 1342 was quantified in monkey serum based on MS analysis of the signature peptide SEQ ID NO: 5. The steps of the assay are disclosed in details the materials and methods section. Briefly, bispecific antibody spiked in human or monkey serum was immunopurified using biotinylated anti-idiotype antibody and streptavidin coated immunomagnetic beads. Bispecific antibody internal standard (IS; stable-isotope-labeled (SIL) signature peptide) was added to immunopurified bispecific antibody before pretreatment with TCEP and iodoacetamide and trypsin digestion. Trypsin digest was then subjected to 2D Trap-Nano LC-Nano ESI MS/MS using Thermo Q-Exactive Orbitrap Mass Spectrometer.

### GBR 1302 Quantification in Human Serum

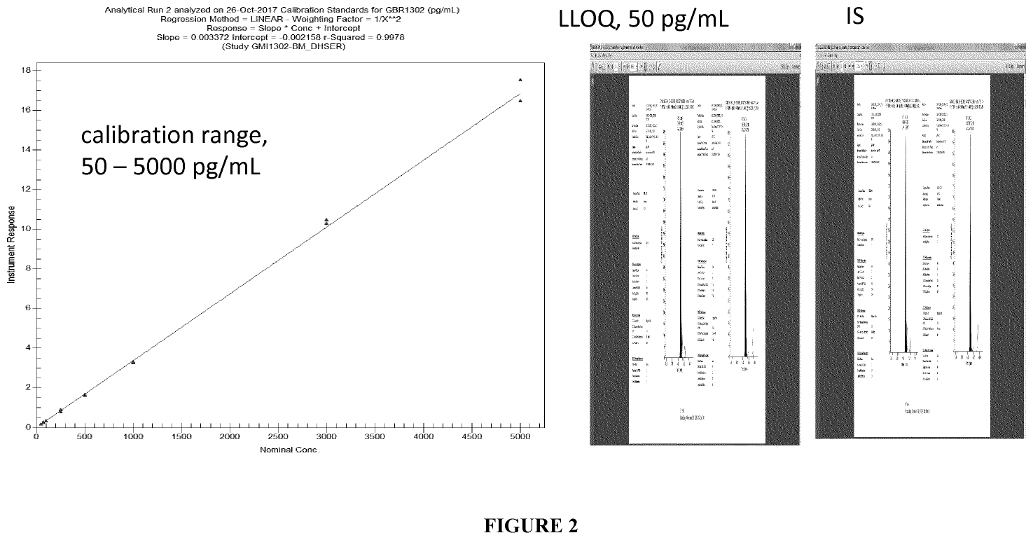

A linear calibration curve was established with a mean correlation coefficient of R²=(0.9978) using 8 standards (STD1 to STD8: 50, 75, 100, 250, 500, 1000, 3000 and 5000 pg/mL). Calibration curve was linear for 2 orders of magnitude and gave a LLOQ of 50 pg/mL (FIG. 2). GBR 1302 concentrations derived using the linear regression curves generated in these experiments displayed an accuracy of (97.8-100.9%) and a % CV (imprecision)<9.4, FIG. 2 and Table 1.

TABLE 1
GBR 1302 quantification in human serum: accuracy and precision

| | STD 1 | STD 2 | STD 3 | STD 4 | STD 5 | STD 6 | STD 7 | STD 8 |
|---|---|---|---|---|---|---|---|---|
| Theor. Conc. | 50 | 75 | 100 | 250 | 500 | 1000 | 3000 | 5000 |
| Found Conc. pg/mL | | | | | | | | |
| #1 | 48.46 | 76.14 | 98.42 | 269.95 | 480.97 | 986.58 | 3108.91 | 4890.80 |
| #2 | 51.89 | 74.45 | 99.29 | 236.42 | 494.30 | 970.26 | 3060.55 | 5202.19 |
| Mean | 50.18 | 75.30 | 98.86 | 253.19 | 487.63 | 978.42 | 3084.73 | 5046.50 |
| S.D. | 2.42 | 1.19 | 0.62 | 23.71 | 9.43 | 11.54 | 34.20 | 220.19 |
| % CV | 4.8 | 1.6 | 0.6 | 9.4 | 1.9 | 1.2 | 1.1 | 4.4 |
| % Theoretical | 100.4 | 100.4 | 98.9 | 101.3 | 97.5 | 97.8 | 102.8 | 100.9 |
| % Dev | 0.4 | 0.4 | −1.1 | 1.3 | −2.5 | −2.2 | 2.8 | 0.9 |
| n | 2 | 2 | 2 | 2 | 2 | 2 | 2 | 2 |

### GBR 1342 Quantification in Monkey Serum

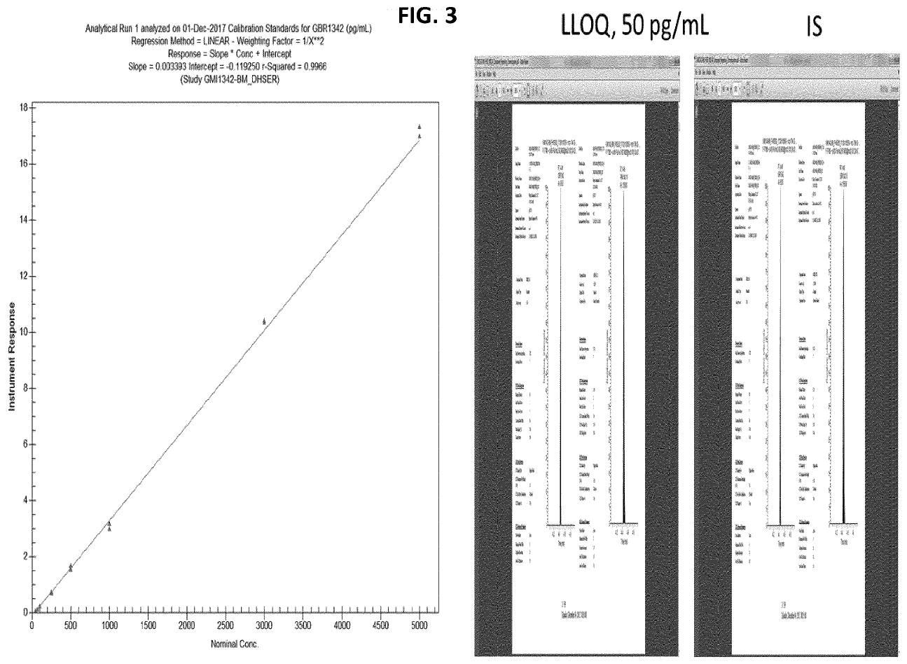

A linear calibration curve was established with a mean correlation coefficient of R²=(0.9966) using 8 standards (STD1 to STD8: 50, 75, 100, 250, 500, 1000, 3000 and 5000 pg/mL). Calibration curve was linear for 2 orders of magnitude and gave a LLOQ of 50 pg/mL (FIG. 3). GBR 1342 concentrations derived using the linear regression curves generated in these experiments displayed an accuracy of (94.3-101.8%) and a % CV (imprecision)<5.7, FIG. 3 and Table 2.

TABLE 2
GBR 1342 quantification in monkey serum: accuracy and precision

| | Std 1 | Std 2 | Std 3 | Std 4 | Std 5 | Std 6 | Std 7 | Std 8 |
|---|---|---|---|---|---|---|---|---|
| Theor. Conc. | 50 | 75 | 100 | 250 | 500 | 1000 | 3000 | 5000 |
| Found Conc. | | | | | | | | |
| #1 | 53.66 | 75.20 | 97.11 | 255.77 | 529.56 | 915.63 | 3103.21 | 5144.84 |
| #2 | 46.56 | 75.18 | 102.49 | 238.59 | 486.42 | 969.49 | 3087.14 | 5038.80 |
| Mean | 50.11 | 75.19 | 99.80 | 247.18 | 507.99 | 942.56 | 3095.18 | 5091.82 |
| S.D. | 5.02 | 0.02 | 3.81 | 12.15 | 30.50 | 38.08 | 11.36 | 74.98 |
| % CV | 10 | 0 | 3.8 | 4.9 | 6 | 4 | 0.4 | 1.5 |
| % Theoretical | 100.2 | 100.3 | 99.8 | 98.9 | 101.6 | 94.3 | 103.2 | 101.8 |
| % Dev | 0.2 | 0.3 | −0.2 | −1.1 | 1.6 | −5.7 | 3.2 | 1.8 |
| n | 2 | 2 | 2 | 2 | 2 | 2 | 2 | 2 |

These results show that the LC-HRMS/MS assay according to the present invention can achieve bispecific antibody quantification in human and non-human primate serum with a high sensitivity (LLOQ of 100 pg/mL), a wide range of detection (two orders of magnitude, 50 pg/mL to 5000 pg/mL) and good precision and accuracy. Consequently, the LC-HRMS/MS assay according to the present invention is a very performant assay for preclinical and clinical studies of bispecific antibody therapeutics.

### Example 3: Pharmacokinetics Studies of GBR1302 in Patients with Progressive HER2-Positive Solid Tumors

### Material and Methods

To evaluate the pharmacokinetic of GBR1302 in adults with progressive HER2-positive solid tumors for which no standard or curative treatment is available, a phase 1, first-in-human, open-label, multicenter, dose-escalation study was carried. Subjects received intravenous GBR 1302 on Day 1 and Day 15 in 28-day treatment cycles at escalating dose levels, starting at 1 ng/kg. The first 4 cohorts consisted of a single subject; subsequent cohorts are being enrolled using a 3+3 design. Blood samples were collected for pharmacokinetic (PK) and antidrug antibody (ADA) analyses (secondary endpoints). Quantification of GBR 1302 serum concentrations (for PK) and detection/confirmation of anti GBR 1302 antibodies (for immunogenicity) were performed using validated LC/MS/MS and ELISA methods, respectively. PK parameters were evaluated using standard non-compartmental methods.

The following PK parameters were estimated:
Maximum observed serum concentration (Cₘₐₓ)
Area under the serum concentration-time curve from time 0 to time of the last measurable concentration (AUCₗₐₛₜ)
Time of maximum observed serum concentration (Tₘₐₓ)
Time of last observed serum concentration (Tₗₐₛₜ)
Serum elimination half-life (t_{1/2})
Last measurable plasma concentration (Cₗₐₛₜ)

To assess immunogenicity antidrug antibody (ADA) response was measured.

### Results:

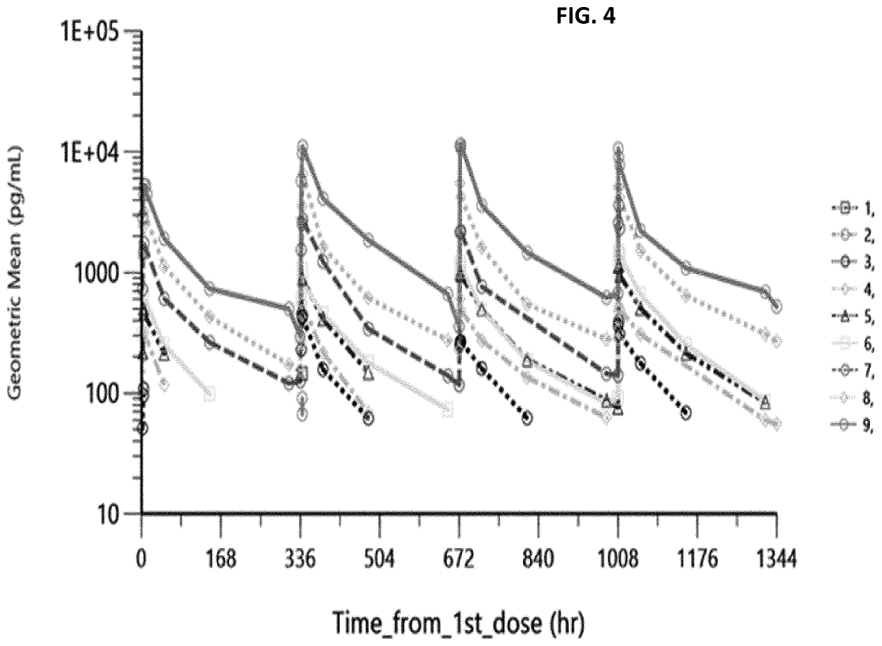

Pharmacokinetic of GBR1302 was studied in 31 subjects over a dose range of 1 ng/kg to 750 ng/kg, as shown in Tables 3 and 4, and FIG. 4.

TABLE 3
Individual subject PK. NCA analysis using Phoenix WinNonlin version 8.0 linear trapezoidal
method with IV infusion dosing. For PK analysis, actual infusion start and end times were
used along with actual elapsed PK sampling time points upto cohort 8. For cohort 9, actual PK
sampling times were not available, hence scheduled sampling time were used for PK calculations.

| CHRT | SUBJ | Dosing_ | Dose | Cₘₐₓ | AUCₗₐₛₜ | Tₘₐₓ | Tₗₐₛₜ | t_{1/2} | Cₗₐₛₜ |
|---|---|---|---|---|---|---|---|---|---|
| ID | ID | Occasion | (ng/kg) | (ng/mL) | (hr*ng/mL) | (hr) | (hr) | (hr) | (ng/mL) |
| 1 | 102- | Dose 1 | 1 | NC | NC | NC | NC | NC | NC |
| | 001 | | | | | | | | |
| 1 | 102- | Dose 2 | 3 | 0.145 | NC | 4 | 4 | NC | NC |
| | 001 | | | | | | | | |
| 2 | 101- | Dose 1 | 3 | NC | NC | NC | NC | NC | NC |
| | 001 | | | | | | | | |
| 2 | 101- | Dose 2 | 10 | 0.0911 | NC | 4 | 4 | NC | NC |
| | 001 | | | | | | | | |
| 2 | 101- | Dose 3 | 10 | NC | NC | NC | NC | NC | NC |
| | 001 | | | | | | | | |
| 3 | 102- | Dose 1 | 10 | 0.109 | NC | 4.01 | 4.01 | NC | NC |
| | 002 | | | | | | | | |
| 3 | 102- | Dose 2 | 30 | 0.433 | 24.4 | 4.01 | 144 | NC | 0.062 |
| | 002 | | | | | | | | |
| 3 | 102- | Dose 3 | 30 | 0.269 | 21.1 | 4.00 | 146.17 | NC | 0.0623 |
| | 002 | | | | | | | | |
| 3 | 102- | Dose 4 | 30 | 0.395 | 24.0 | 2.00 | 146.67 | ^{b}67.2 | 0.069 |
| | 002 | | | | | | | | |
| 4 | 101- | Dose 1 | 30 | 0.32 | 10.0 | 4.07 | 48.00 | NC | 0.117 |
| | 002 | | | | | | | | |
| 4 | 101- | Dose 2 | 60 | 0.559 | 31.8 | 4.07 | 143.97 | NC | 0.0689 |
| | 002 | | | | | | | | |
| 4 | 101- | Dose 3 | 60 | 0.606 | 54.4 | 2.22 | 311.72 | 127 | 0.0627 |
| | 002 | | | | | | | | |
| 4 | 101- | Dose 4 | 60 | 0.528 | 69 | 2.00 | 335.02 | 114 | 0.0558 |
| | 002 | | | | | | | | |
| 5 | 101- | Dose 1 | 60 | 0.327 | 10.7 | 4.02 | 48.08 | NC | 0.129 |
| | 003 | | | | | | | | |
| 5 | 101- | Dose 1 | 60 | 0.362 | 11.9 | 4.05 | 49.22 | NC | 0.134 |
| | 004 | | | | | | | | |
| 5 | 101- | Dose 1 | 60 | 0.602 | 18.6 | 4.28 | 46.92 | NC | 0.229 |
| | 005 | | | | | | | | |
| 5 | 101- | Dose 1 | 60 | 0.403 | 30.7 | 4.03 | 143.67 | NC | 0.0786 |
| | 006 | | | | | | | | |
| 5 | 102- | Dose 1 | 60 | 0.545 | 54.5 | 4.00 | 312.08 | 123 | 0.053 |
| | 004 | | | | | | | | |
| 5 | 103- | Dose 1 | 60 | 0.63 | 18.9 | 4.08 | 48 | NC | 0.169 |
| | 001 | | | | | | | | |
| 5 | 103- | Dose 1 | 60 | 0.468 | 27.1 | 4.02 | 144.03 | NC | 0.0659 |
| | 002 | | | | | | | | |
| 5 | 104- | Dose 1 | 60 | 0.884 | 87.6 | 4.08 | 316.25 | 92.8 | 0.0673 |
| | 002 | | | | | | | | |
| 5 | 101- | Dose 2 | 100 | 0.575 | 32.4 | 4.08 | 143.53 | NC | 0.0663 |
| | 003 | | | | | | | | |
| 5 | 101- | Dose 2 | 100 | 0.726 | 60 | 4.07 | 313.2 | 114 | 0.0585 |
| | 004 | | | | | | | | |
| 5 | 101- | Dose 2 | 100 | 0.789 | 53.5 | 4.08 | 144 | NC | 0.123 |
| | 006 | | | | | | | | |
| 5 | 102- | Dose 2 | 100 | 1.08 | 105 | 4 | 359.17 | ^{b}150 | 0.0803 |
| | 004 | | | | | | | | |
| 5 | 103- | Dose 2 | 100 | 0.821 | 64.5 | 4.27 | 335.23 | 115 | 0.0586 |
| | 002 | | | | | | | | |
| 5 | 104- | Dose 2 | 100 | 1.78 | 186 | 4.15 | 335.5 | 98.1 | 0.0931 |
| | 002 | | | | | | | | |
| 5 | 101- | Dose 3 | 100 | 0.669 | 64 | 2 | 335.37 | 119 | 0.0512 |
| | 004 | | | | | | | | |
| 5 | 101- | Dose 3 | 100 | 0.867 | 73.3 | 2.08 | 315.17 | 85.8 | 0.0584 |
| | 006 | | | | | | | | |
| 5 | 102- | Dose 3 | 100 | 0.899 | 98.2 | 2 | 335.67 | ^{b}150 | 0.0875 |
| | 004 | | | | | | | | |
| 5 | 103- | Dose 3 | 100 | 1.04 | 77.3 | 4 | 335.73 | ^{b}177 | 0.0624 |
| | 002 | | | | | | | | |
| 5 | 104- | Dose 3 | 100 | 1.98 | 204 | 2 | 360.08 | 116 | 0.121 |
| | 002 | | | | | | | | |
| 5 | 101- | Dose 4 | 100 | 0.832 | 63.5 | 1.03 | 312.6 | 92.7 | 0.0624 |
| | 004 | | | | | | | | |
| 5 | 102- | Dose 4 | 100 | 1.02 | 79.5 | 2 | 335.67 | 117 | 0.0618 |
| | 004 | | | | | | | | |
| 5 | 103- | Dose 4 | 100 | 1.33 | 83.4 | 1.03 | 335.27 | 84.6 | 0.072 |
| | 002 | | | | | | | | |
| 5 | 104- | Dose 4 | 100 | 2.92 | 191 | 1 | 287.42 | 95.4 | 0.181 |
| | 002 | | | | | | | | |
| 6 | 101- | Dose 1 | 100 | 0.666 | 38 | 4.12 | 142.67 | NC | 0.103 |
| | 007 | | | | | | | | |
| 6 | 103- | Dose 1 | 100 | 0.533 | 28.1 | 4.07 | 119.65 | NC | 0.0751 |
| | 005 | | | | | | | | |
| 6 | 201- | Dose 1 | 100 | 0.733 | 58.7 | 4.2 | 313.08 | 125 | 0.0611 |
| | 001 | | | | | | | | |
| 6 | 101- | Dose 2 | 200 | 1.19 | 107 | 4.08 | 314.87 | 119 | 0.101 |
| | 007 | | | | | | | | |
| 6 | 103- | Dose 2 | 200 | 0.929 | 79.2 | 6.8 | 292.8 | NC | 0.0598 |
| | 005 | | | | | | | | |
| 6 | 201- | Dose 2 | 200 | 1.05 | 83.4 | 4.1 | 337.4 | 94.5 | 0.0695 |
| | 001 | | | | | | | | |
| 6 | 101- | Dose 3 | 200 | 1.15 | 94.9 | 2.03 | 335.08 | ^{b}154 | 0.0754 |
| | 007 | | | | | | | | |
| 6 | 103- | Dose 3 | 200 | 1.02 | 67.9 | 4 | 335.6 | 123 | 0.065 |
| | 005 | | | | | | | | |
| 6 | 201- | Dose 3 | 200 | 1.94 | 146 | 3.92 | 336.85 | 104 | 0.111 |
| | 001 | | | | | | | | |
| 6 | 101- | Dose 4 | 200 | 1.39 | 78.8 | 2 | 141.33 | ^{b}67.5 | 0.261 |
| | 007 | | | | | | | | |
| 6 | 201- | Dose 4 | 200 | 1.68 | 136 | 4.27 | 315.78 | 86.9 | 0.0864 |
| | 001 | | | | | | | | |
| 7 | 101- | Dose 1 | 200 | 2.17 | 126 | 4.67 | 333.93 | 126 | 0.0675 |
| | 009 | | | | | | | | |
| 7 | 101- | Dose 1 | 200 | 1.39 | 102 | 5.75 | 333.67 | ^{b}117 | 0.12 |
| | 010 | | | | | | | | |
| 7 | 103- | Dose 1 | 200 | 2.17 | 224 | 4.07 | 286.43 | ^{b}138 | 0.246 |
| | 006 | | | | | | | | |
| 7 | 101- | Dose 2 | 300 | 2.54 | 184 | 4.65 | 315 | 95.1 | 0.13 |
| | 009 | | | | | | | | |
| 7 | 101- | Dose 2 | 300 | 2.44 | 368 | 4.03 | 530.63 | 113 | 0.0876 |
| | 010 | | | | | | | | |
| 7 | 103- | Dose 2 | 300 | 3.05 | 322 | 4 | 382.97 | 109 | 0.156 |
| | 006 | | | | | | | | |
| 7 | 101- | Dose 3 | 300 | 1.93 | 325 | 2.03 | 334.83 | NC | 0.141 |
| | 010 | | | | | | | | |
| 7 | 103- | Dose 3 | 300 | 2.42 | 143 | 2 | 95.72 | NC | 0.765 |
| | 006 | | | | | | | | |
| 7 | 101- | Dose 4 | 300 | 3.62 | NC | 2 | 4.07 | NC | NC |
| | 010 | | | | | | | | |
| 8 | 101- | Dose 1 | 300 | 3.21 | 273 | 6.22 | 334.92 | ^{b}127 | 0.198 |
| | 011 | | | | | | | | |
| 8 | 101- | Dose 1 | 300 | 2.45 | 182 | 4.02 | 333.17 | 108 | 0.123 |
| | 012 | | | | | | | | |
| 8 | 201- | Dose 1 | 300 | 2.02 | 224 | 4.18 | 335.53 | ^{b}139 | 0.154 |
| | 002 | | | | | | | | |
| 8 | 101- | Dose 2 | 500 | 6.59 | 495 | 6.37 | 336.95 | 126 | 0.302 |
| | 011 | | | | | | | | |
| 8 | 101- | Dose 2 | 500 | 4.45 | 332 | 4.03 | 336.35 | ^{b}130 | 0.205 |
| | 012 | | | | | | | | |
| 8 | 201- | Dose 2 | 500 | 2.54 | 216 | 4.18 | 335.52 | ^{b}165 | 0.194 |
| | 002 | | | | | | | | |
| 8 | 101- | Dose 3 | 500 | 5.68 | 436 | 2.5 | 335.22 | ^{b}153 | 0.377 |
| | 011 | | | | | | | | |
| 8 | 101- | Dose 3 | 500 | 6.23 | 299 | 2.03 | 335.88 | ^{b}142 | 0.215 |
| | 012 | | | | | | | | |
| 8 | 201- | Dose 3 | 500 | 4.71 | 265 | 2 | 311.93 | ^{b}85.5 | 0.268 |
| | 002 | | | | | | | | |
| 8 | 101- | Dose 4 | 500 | 6.92 | 402 | 2 | 338.37 | ^{b}146 | 0.334 |
| | 011 | | | | | | | | |
| 8 | 101- | Dose 4 | 500 | 5.43 | 278 | 1.03 | 336.58 | ^{b}165 | 0.217 |
| | 012 | | | | | | | | |
| 9 | 101- | Dose 1 | 500 | 7.05 | 626 | 4.02 | 334.08 | ^{b}197 | 0.661 |
| | 013 | | | | | | | | |
| 9 | 101- | Dose 1 | 500 | 4.46 | 377 | 11.28 | 360.2 | 145 | 0.226 |
| | 014 | | | | | | | | |
| 9 | 106- | Dose 1 | 500 | 3.14 | 199 | 4 | 143.83 | NC | 0.6 |
| | 001 | | | | | | | | |
| 9 | 106- | Dose 1 | 500 | 5.51 | 321 | 6 | 334.83 | 117 | 0.161 |
| | 002 | | | | | | | | |
| 9 | 107- | Dose 1 | 500 | 6.13 | 287 | 5.17 | 168.92 | NC | 0.181 |
| | 001 | | | | | | | | |
| 9 | 201- | Dose 1 | 500 | 6.24 | 346 | 4.02 | 143.87 | NC | 0.877 |
| | 004 | | | | | | | | |
| 9 | 203- | Dose 1 | 500 | 5.01 | 359 | 9.55 | 335.27 | 105 | 0.216 |
| | 001 | | | | | | | | |
| 9 | 204- | Dose 1 | 500 | 5.24 | 170 | 8.4 | 49.45 | NC | 1.87 |
| | 001 | | | | | | | | |
| 9 | 204- | Dose 1 | 500 | 7.88 | 784 | 3.93 | 335.5 | 155 | 0.813 |
| | 002 | | | | | | | | |
| 9 | 204- | Dose 1 | 500 | 8.02 | 727 | 4.02 | 336.58 | 94.3 | 0.321 |
| | 003 | | | | | | | | |
| 9 | 209- | Dose 1 | 500 | 7.94 | 423 | 4.33 | 146.17 | NC | 0.802 |
| | 001 | | | | | | | | |
| 9 | 209- | Dose 1 | 500 | 4.04 | 132 | 2.05 | 48.22 | NC | 1.49 |
| | 002 | | | | | | | | |
| 9 | 209- | Dose 1 | 500 | 13.2 | 898 | 5.15 | 260.23 | 74.8 | 0.648 |
| | 003 | | | | | | | | |
| 9 | 209- | Dose 1 | 500 | 4.55 | 253 | 4 | 336.73 | ^{b}208 | 0.143 |
| | 006 | | | | | | | | |
| 9 | 209- | Dose 1 | 500 | 5.39 | 266 | 7.92 | 145.92 | NC | 0.642 |
| | 007 | | | | | | | | |
| 9 | 101- | Dose 2 | 750 | 16.2 | 861 | 4.03 | 146.08 | NC | 2.24 |
| | 013 | | | | | | | | |
| 9 | 101- | Dose 2 | 750 | 9.14 | 576 | 4.02 | 289.33 | 115 | 0.441 |
| | 014 | | | | | | | | |
| 9 | 106- | Dose 2 | 750 | 7.56 | 242 | 5.92 | 48 | NC | 2.87 |
| | 002 | | | | | | | | |
| 9 | 203- | Dose 2 | 750 | 8.51 | 577 | 4.17 | 360.68 | 138 | 0.373 |
| | 001 | | | | | | | | |
| 9 | 204- | Dose 2 | 750 | 12.4 | 954 | 3.98 | 337.02 | ^{b}160 | 0.895 |
| | 002 | | | | | | | | |
| 9 | 204- | Dose 2 | 750 | 13.3 | 1290 | 4.02 | 309.98 | 111 | 1.26 |
| | 003 | | | | | | | | |
| 9 | 209- | Dose 2 | 750 | 20.8 | 1490 | 4.05 | 526.05 | 104 | 0.137 |
| | 003 | | | | | | | | |
| 9 | 209- | Dose 2 | 750 | 4.28 | NC | 4.02 | 4.02 | NC | NC |
| | 006 | | | | | | | | |
| 9 | 203- | Dose 3 | 750 | 10.7 | 318 | 3.17 | 46.27 | NC | 3.34 |
| | 001 | | | | | | | | |
| 9 | 204- | Dose 3 | 750 | 14.4 | 824 | 2 | 335.6 | ^{b}141 | 0.672 |
| | 002 | | | | | | | | |
| 9 | 209- | Dose 3 | 750 | 13.3 | 884 | 4.28 | 312.75 | 81 | 0.44 |
| | 003 | | | | | | | | |
| 9 | 204- | Dose 4 | 750 | 10.6 | 590 | 1.02 | 360.52 | 149 | 0.522 |
| | 002 | | | | | | | | |

^{b}flagged because the either the R2 adjusted is <0.8, and/or AUC % extrapolated is >20%, and/or duration of Kel estimation is <1.5-fold of the resultant t_{1/2}.
^{b}flagged because the either the R2 adjusted is <0.8, and/or AUC % extrapolated is >20%, and/or duration of Kel estimation is <1.5-fold of the resultant t1/2

TABLE 4
Summary PK parameters. [Mean (SD)] of GBR 1302.

| Summary PK parameters | | | | | | | | |
|---|---|---|---|---|---|---|---|---|
| [Mean (SD)] of GBR 1302 | | | | | | | | |
| Cohort | Dosing_ | Dose | Cₘₐₓ | AUCₗₐₛₜ | #Tₘₐₓ | Tₗₐₛₜ | t_{1/2} | Cₗₐₛₜ |
| ID | Occasion | (ng/kg) | (ng/mL) | (hr*ng/mL) | (hr) | (hr) | (hr) | (ng/mL) |
| Cohort 1 | Dose 1 | 1 | NC | NC | NC | NC | NC | NC |
| (N = 1) | | | | | | | | |
| Cohort 1 | Dose 2 | 3 | 0.145 | NC | 4.00 | 4.00 | NC | NC |
| (N = 1) | | | | | | | | |
| Cohort 2 | Dose 1 | 3 | NC | NC | NC | NC | NC | NC |
| (N = 1) | | | | | | | | |
| Cohort 2 | Dose 2 | 10 | 0.091 | NC | 4.00 | 4.00 | NC | NC |
| (N = 1) | | | | | | | | |
| Cohort 2 | Dose 3 | 10 | NC | NC | NC | NC | NC | NC |
| (N = 1) | | | | | | | | |
| Cohort 3 | Dose 1 | 10 | 0.109 | NC | 4.01 | 4.01 | NC | NC |
| (N = 1) | | | | | | | | |
| Cohort 3 | Dose 2 | 30 | 0.433 | 24.4 | 4.01 | 144.00 | NC | 0.062 |
| (N = 1) | | | | | | | | |
| Cohort 3 | Dose 3 | 30 | 0.269 | 21.1 | 4.00 | 146.17 | NC | 0.062 |
| (N = 1) | | | | | | | | |
| Cohort 3 | Dose 4 | 30 | 0.395 | 24.0 | 2.00 | 146.67 | NC | 0.069 |
| (N = 1) | | | | | | | | |
| Cohort 4 | Dose 1 | 30 | 0.32 | 10.0 | 4.07 | 48.00 | NC | 0.117 |
| (N = 1) | | | | | | | | |
| Cohort 4 | Dose 2 | 60 | 0.559 | 31.8 | 4.07 | 143.97 | NC | 0.069 |
| (N = 1) | | | | | | | | |
| Cohort 4 | Dose 3 | 60 | 0.606 | 54.4 | 2.22 | 311.72 | 127 | 0.063 |
| (N = 1) | | | | | | | | |
| Cohort 4 | Dose 4 | 60 | 0.528 | 69.0 | 2.00 | 335.02 | 114 | 0.056 |
| (N = 1) | | | | | | | | |
| Cohort 5 | Dose 1 | 60 | 0.528 | 32.5 | 4.04 | 96.45 | ^{a}108 | 0.116 |
| (N = 8) | | | (0.181) | (26.3) | (4.00-4.28) | (46.92-316.25) | (21.4) | (0.061) |
| Cohort 5 | Dose 2 | 100 | 0.962 | 83.6 | 4.08 | 324.22 | ^{b}109 | 0.080 |
| (N = 6) | | | (0.433) | (55.5) | (4.00-4.27) | (143.53-359.17) | (9.48) | (0.025) |
| Cohort 5 | Dose 3 | 100 | 1.09 | 103 | 2.00 | 335.67 | ^{b}107 | 0.076 |
| (N = 5) | | | (0.514) | (57.6) | (2.00-4.00) | (315.17-360.08) | (18.4) | (0.029) |
| Cohort 5 | Dose 4 | 100 | 1.53 | 104 | 1.03 | 323.94 | 97.4 | 0.094 |
| (N = 4) | | | (0.952) | (58.4) | (1.00-2.00) | (287.42-335.67) | (13.8) | (0.058) |
| Cohort 6 | Dose 1 | 100 | 0.644 | 41.6 | 4.12 | 142.67 | ^{c}125 | 0.080 |
| (N = 3) | | | (0.102) | (15.6) | (4.07-4.20) | (119.65-313.08) | | (0.021) |
| Cohort 6 | Dose 2 | 200 | 1.06 | 89.9 | 4.10 | 314.87 | ^{a}107 | 0.077 |
| (N = 3) | | | (0.131) | (15.0) | (4.08-6.8) | (292.8-337.4) | (17.3) | (0.022) |
| Cohort 6 | Dose 3 | 200 | 1.37 | 103 | 3.92 | 335.60 | ^{a}114 | 0.084 |
| (N = 3) | | | (0.498) | (39.7) | (2.03-4.00) | (335.08-336.85) | (13.4) | (0.024) |
| Cohort 6 | Dose 4 | 200 | 1.54 | 107 | 3.14 | 228.56 | ^{c}86.9 | 0.174 |
| (N = 2) | | | (0.205) | (40.4) | (2.00-4.27) | (141.33-315.78) | | (0.123) |
| Cohort 7 | Dose 1 | 200 | 1.91 | 151 | 4.67 | 333.67 | ^{c}126 | 0.145 |
| (N = 3) | | | (0.45) | (64.6) | (4.07-5.75) | (286.43-333.93) | | (0.092) |
| Cohort 7 | Dose 2 | 300 | 2.68 | 291 | 4.03 | 382.97 | 106 | 0.125 |
| (N = 3) | | | (0.327) | (95.8) | (4.00-4.65) | (315-530.63) | (9.39) | (0.035) |
| Cohort 7 | Dose 3 | 300 | 2.18 | 234 | 2.02 | 215.28 | NC | 0.453 |
| (N = 2) | | | (0.346) | (129) | (2.00-2.03) | (95.72-334.83) | | (0.441) |
| Cohort 7 | Dose 4 | 300 | 3.62 | NC | 2.00 | 4.07 | NC | 2.32 |
| (N = 1) | | | | | | | | |
| Cohort 8 | Dose 1 | 300 | 2.56 | 226 | 4.18 | 334.92 | ^{a}108 | 0.158 |
| (N = 3) | | | (0.603) | (45.6) | (4.02-6.22) | (333.17-335.53) | | (0.038) |
| Cohort 8 | Dose 2 | 500 | 4.53 | 348 | 4.18 | 336.35 | ^{a}126 | 0.234 |
| (N = 3) | | | (2.03) | (140) | (4.03-6.37) | (335.52-336.95) | | (0.059) |
| Cohort 8 | Dose 3 | 500 | 5.54 | 333 | 2.03 | 335.22 | NC | 0.287 |
| (N = 3) | | | (0.770) | (90.5) | (2.00-2.50) | (311.93-335.88) | | (0.083) |
| Cohort 8 | Dose 4 | 500 | 6.18 | 340 | 1.52 | 337.48 | NC | 0.276 |
| (N = 2) | | | (1.05) | (87.7) | (1.03-2.00) | (336.58-338.37) | | (0.083) |
| Cohort 9 | Dose 1 | 500 | 6.25 | 411 | 4.33 | 260.23 | ^{d}115 | 0.643 |
| (N = 15) | | | (2.42) | (236) | (2.05-11.28) | (48.22-360.2) | (30.5) | (0.499) |
| Cohort 9 | Dose 2 | 750 | 11.5 | ^{e}856 | 4.03 | 299.66 | ^{f}117 | ^{e}1.56 |
| (N = 8) | | | (5.28) | (435) | (3.98-5.92) | (4.02-526.05) | (14.7) | (1.45) |
| Cohort 9 | Dose 3 | 750 | 12.8 | 675 | 3.17 | 312.75 | ^{c}81 | 1.48 |
| (N = 3) | | | (1.90) | (311) | (2.00-4.28) | (46.27-335.6) | | (1.61) |
| Cohort 9 | Dose 4 | 750 | 10.6 | 590 | 1.02 | 360.52 | 149 | 0.522 |
| (N = 1) | | | | | | | | |

NC: Not Calculable;
#Median (Min-Max);
^{a}N = 2;
^{b}N = 3;
^{c}N = 1;
^{d}N = 6;
^{e}N = 7;
^{f}N = 4.

Serum concentrations were less than the lower limit of quantification of 50 pg/mL at the first dose (1 ng/kg), and only transient concentrations were observed at 3 and 10 ng/kg dose levels. Evaluable PK profiles were observed from 30 ng/kg onwards. GBR 1302 showed maximum plasma concentration (Cₘₐₓ) around the end of infusion, after which serum concentrations declined bi-exponentially with a mean terminal half-life of around 4 to 7 days. Both Cₘₐₓ and area under the curve (AUC₀₋₄) showed a near dose-proportional increase up to 750 ng/kg (maximum evaluated dose). None of the samples collected from subjects up to cohort 5 showed positive ADA response. These results show a favorable, linear PK, and none of the subjects evaluated so far showed positive ADA response.

1. A method for quantifying a bispecific antibody in a biological sample, wherein the antibody comprises an engineered human IgG CH3 heterodimer comprising several substitutions in the CH3 domains, including at least two substitutions at positions 80 to 88 of a first CH3 domain, wherein the method comprises quantifying a signature peptide of said antibody by mass spectrometry, wherein the signature peptide is a tryptic peptide corresponding to positions 80 to 88 of said first CH3 domain, and wherein the amino acid positions are indicated according to IGMT® numbering.

2. The method according to claim 1, wherein the signature peptide consists of a sequence:
(SEQ ID NO: 1)
TX₁PPX₂LX₃SX₄GSFX₅LX₆SX₇
wherein X₁ represents T or D, X₂ represents V, L, P or M, X₃ represents D, Q or E, X₄ represents D or Q, X₅ represents F, A or W, X₆ represents S, W or H, and X₇ represents K or R, with the proviso that when X₁ is T, then at least one of X₂, X₃, X₄, X₅, and X₇ is such that X₂ is L, P or M; X₃ is Q or E; X₄ is Q; X₅ is A or W; and X₇ is R.

3. The method according to claim 2, wherein the signature peptide is selected from the group consisting of: TTPPVLDSDGSFALSSK (SEQ ID NO: 3), TDPPLLESDGSFALSSR (SEQ ID NO: 4), TDPPLLESQGSFALSSR (SEQ ID NO: 5), TTPPPLQSDGSFWLWSK (SEQ ID NO: 6) and TTPPMLESDGSFFLHSK (SEQ ID NO: 7), preferably SEQ ID NO: 4 or SEQ ID NO: 5.

4. The method according to any one of the preceding claims, wherein the bispecific antibody comprises a human IgG CH3 domain heterodimer engineered using T-cell receptor-based immunoglobulin domain interface, wherein the first CH3 domain is from human IgG1 and comprises at least the substitutions F85.1A and Y86S and the second CH3 domain is from human IgG1 or IgG3 and comprises at least the substitutions S20K, T22V, K26T, K79Y, K88W and T90N, and wherein said positions are indicated according to IGMT® numbering.

5. The method according to claim 4, wherein:
the first CH3 domain further comprises one or more of the following substitutions: Q3E, Y5A, L7F, S20T, T22V, K26T, T81D, V84L, D84.2E, K88R and T90R; preferably Q3E, Y5A, L7F, S20T, T22V, K26T, T81D, V84L, D84.2E, K88R and T90R or Q3E, Y5A, L7F, S20T, T22V, K26T, T81D, V84L, D84.2E and K88R; and
the second CH3 domain further comprises one or more of the following substitutions: Q3A, D12E, L14M, N44S, V84M, F85.1S, Y86V, V101I, H115R and Y116F; preferably F85.1S and Y86V; F85.1S, Y86V and Q3A for a CH3 domain from human IgG1, and D12E, L14M, N44S, V84M, F85.1S, Y86V, V101I, H115R and Y116F fora CH3 domain from human IgG3.

6. The method according to any one of the preceding claims, wherein the antibody comprises a human IgG CH3 domain heterodimer engineered using immunoglobulin domain interface exchange between human IgG and IgD CH3 domains, wherein the first CH3 domain comprises the substitutions: Q3V, Y5L, K26S, V84P, D84.2Q, F85.1W and Y86W and the second CH3 domain comprises the substitutions: S20W, K79A, T81A, K88V and T90R.

7. The method according to any one of the preceding claims, wherein the antibody comprises a human IgG CH3 domain heterodimer engineered using immunoglobulin domain interface exchange between human IgG and IgM CH3 domains, wherein the first CH3 domain comprises the substitutions: Q3D, K26T, V84M, D84.2E and Y86H and the second CH3 domain comprises the substitutions: S20T, K79V, T81S and K88I.

8. The method according to any one of the preceding claims, wherein the bispecific antibody comprises a Fc, a Fab and a scFv from human immunoglobulin.

9. The method according to any one of the preceding claims, wherein the bispecific antibody is a therapeutic antibody.

10. The method according to any one of the preceding claims, wherein the bispecific antibody is a bispecific anti-CD3 antibody, preferably anti-CD3 and anti-Her2, anti-CD3 and anti-CD38 or anti-CD3 and anti-EGFR bispecific antibody.

11. The method according to any one of the preceding claims, which comprises the steps of:
a) purifying the bispecific antibody from the biological sample by immunocapture,
b) digesting the bispecific antibody obtained in step a) with trypsin or trypsin/Lys C to generate peptides comprising the signature peptide, and
c) subjecting the peptides obtained in step b) to mass spectrometry to determine the amount of signature peptide in the biological sample by comparison with an internal standard or with the use of calibration standards.

12. The method according to claim 11, wherein the immunocapture is performed with an antibody specific for the bispecific antibody; preferably an anti-idiotype antibody, an antibody against the signature peptide, or a combination thereof; preferably the immunocapture is performed on a solid support or using immunomagnetic separation; more preferably using biotinylated antibody and streptavidin coated magnetic beads.

13. The method according to any one of the preceding claims, wherein the mass spectrometry comprises two-dimensional nano-liquid chromatography coupled to electrospray-ionization Orbitrap mass spectrometry.

14. The method according to any one of the preceding claims, wherein the biological sample is a human biological sample, preferably a human body fluid, more preferably human serum.

15. The method according to any one of the preceding claims, wherein the lower limit of quantification of the bispecific antibody is 50 pg/ml and the detection range of the bispecific antibody is from 50 pg/ml to 5000 pg/ml in human serum.

16. A kit for quantifying a bispecific antibody in a biological sample using the method of any one of the preceding claims, comprising at least a signature peptide as defined in any one of claims 1 to 3; preferably further comprising an antibody specific for the bispecific antibody as defined in claim 12.


SEQUENCE LISTING (16 sequences)

SEQ ID NO: 1 - protein, 17 aa, Artificial Sequence
  Signature peptide 1
  misc_feature (2)..(2): T D
  misc_feature (5)..(5): V, L, P or M
  misc_feature (7)..(7): D, Q or E
  misc_feature (9)..(9): D or Q
  misc_feature (13)..(13): F, A or W
  misc_feature (15)..(15): S, W or H
  misc_feature (17)..(17): K or R
TXPPXLXSXGSFXLXSX

SEQ ID NO: 2 - protein, 17 aa, Artificial Sequence
  Signature peptide 2
  misc_feature (5)..(5): V or M
  misc_feature (17)..(17): K or R
TTPPXLDSDGSFFLYSX

SEQ ID NO: 3 - protein, 17 aa, Artificial Sequence
  Signature peptide 3
TTPPVLDSDGSFALSSK

SEQ ID NO: 4 - protein, 17 aa, Artificial Sequence
  Signature peptide 4
TDPPLLESDGSFALSSR

SEQ ID NO: 5 - protein, 17 aa, Artificial Sequence
  Signature peptide 5
TDPPLLESQGSFALSSR

SEQ ID NO: 6 - protein, 17 aa, Artificial Sequence
  Signature peptide 6
TTPPPLQSDGSFWLWSK

SEQ ID NO: 7 - protein, 17 aa, Artificial Sequence
  Signature peptide 7
TTPPMLESDGSFFLHSK

SEQ ID NO: 8 - protein, 8 aa, Artificial Sequence
  Signature peptide 8
LYSGVPSR

SEQ ID NO: 9 - protein, 9 aa, Artificial Sequence
  Signature peptide 9
FTISADTSK

SEQ ID NO: 10 - protein, 11 aa, Artificial Sequence
  Signature peptide 10
EPEVATFPPSR

SEQ ID NO: 11 - protein, 495 aa, Artificial Sequence
  Herceptin ScFv LALA BTB Fc_Protein
METDTLLLWVLLLWVPGSTGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRGGGGTDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPEVATFPPSRDELTKNQVTLVCLVTGFYPSDIAVEWESNGQPENNYKTDPPLLESDGSFALSSRLRVDKSRWQQGNVFSCSVMHEALHNHYTQKSLSLSPGK

SEQ ID NO: 12 - protein, 469 aa, Artificial Sequence
  OKT3 H11 1133 LALA BTA_Protein
METDTLLLWVLLLWVPGSTGEVQLVESGGGLVQPGGSLRLSCAASGYTFTRYTMHWVRQAPGKGLEWIGYINPSRGYTYYADSVKGRFTLSTDKSKNTAYLQMSSLRAEDTAVYYCARYYDDHYCLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPAVYTLPPSREEMTKNQVKLVCLVTGFYPSDIAVEWESSGQPENNYYTTPPMLDSDGSFSLVSWLNVDKSRWQQGNIFSCSVMHEALHNRFTQKSLSLSPGK

SEQ ID NO: 13 - protein, 233 aa, Artificial Sequence
  OKT3 L8 LC_Protein
METDTLLLWVLLLWVPGSTGDIQLTQSPSSLSASVGDRVTITCRASSSVSYVAWYQQKPGKAPKRWIYDTSKLYSGVPSRFSGSGSGTDYTLTISSLQPEDFATYYCQQWSSNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 234 aa, Artificial Sequence
  GBR1342 light chain sequence (hum9G7_VL_cK)
MRSPAQLLFLLLLWIPGTNADIQMTQSPSSLSASVGDRVTITCQASQDVITSVAWYQQKPGKAPKLLIYSASYRYTGVPSRFSGSGSGTDFTFTISSLQPEDIATYYCQQHYTIPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 470 aa, Artificial Sequence
  GBR1342 heavy chain sequence (h9G7 1133 BTA LALA FTO (dA))
MRSPAQLLFLLLLWIPGTNAQVTLKESGPTLVKPTQTLTLTCTFSGLSLSTSGKGVGWIRQPPGKALEWLAHIWWDDDKRYNPALKSRLTITKDTSKNQVVLTMTNMDPVDTATYYCARIELGRSYVMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPAVYTLPPSREEMTKNQVKLVCLVTGFYPSDIAVEWESSGQPENNYYTTPPMLDSDGSFSLVSWLNVDKSRWQQGNIFSCSVMHEALHNRFTQKSLSLSPG

SEQ ID NO: 16 - protein, 501 aa, Artificial Sequence
  GBR1342 scFv-Fc sequence hSP34_H3K21_W100eY/T29E-W91F-L95T (hSP34v.3) scFv_11_BTB_D401Q_LALA
MRSPAQLLFLLLLWIPGTNAEVQLVESGGGLVQPGGSLRLSCAASGFTFNTYAMNWVRQAPGKGLEWVARIRSKYNNYATYYADSVKGRFTISRDDSKNTLYLQMNSLRAEDTAVYYCVRHGNFGNSYVSYFAYWGQGTTVTVSSGGGGSGGGGSGGGGSEIVVTQSPATLSVSPGERATLSCRSSTGAVTESNYANWVQEKPGQAFRGLIGGANKRAPGVPARFSGSLSGDEATLTISSLQSEDFAVYYCALFYSNTWVFGQGTKLEIKGGGGTDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPEVATFPPSRDELTKNQVTLVCLVTGFYPSDIAVEWESNGQPENNYKTDPPLLESQGSFALSSRLRVDKSRWQQGNVFSCSVMHEALHNHYTQKSLSLSPG